(19) United States
(12) Patent Application Publication
(10) Pub. No.: US 2024/0366432 A1
(43) Pub. Date: Nov. 7, 2024

(54) Nonwoven with Bio Particles and Methods of Making the Same

(71) Applicant: SOLVENTUM INTELLECTUAL PROPERTIES COMPANY, Maplewood, MN (US)

(72) Inventors: Randilynn B. Christensen, Pine Springs, MN (US); Saurabh Batra, Minneapolis, MN (US); Bryan A. Baker, Minneapolis, MN (US)

(73) Assignee: SOLVENTUM INTELLECTUAL PROPERTIES COMPANY, Maplewood, MN (US)

(21) Appl. No.: 18/687,845

(22) PCT Filed: Jul. 28, 2022
(86) PCT No.: PCT/IB2022/057026

§ 371 Date: Feb. 29, 2024

Related U.S. Application Data
(60) Provisional application No. 63/239,746, filed on Sep. 1, 2021.

(51) Int. Cl. A61F 13/01; A61F 13/00; A61L 15/18; A61L 15/24; A61L 15/62; D04H 1/413; D04H 1/4282; D04H 1/4291; D04H 1/4358; D04H 1/72; D04H 3/16

(52) U.S. Cl. CPC A61F 13/01017; A61F 13/00987; A61L 15/18; A61L 15/24; A61L 15/62; D04H 1/413; D04H 1/4282; D04H 1/4291; D04H 1/4358; D04H 1/72; D04H 3/16; A61F 2013/00119; D10B 2321/022; D10B 2321/121; D10B 2509/022

(57) ABSTRACT

An article. The article includes a nonwoven matrix; and bioactive glass particles enmeshed in the nonwoven matrix; wherein the article is a conformable wound dressing. A method of making the article includes flowing molten polymer through a plurality of orifices to form filaments; attenuating the filaments into fibers; directing a stream of bioactive glass particles amidst the filaments or fibers; and collecting a nonwoven matrix and bioactive glass particles enmeshed in the nonwoven matrix.

### BACKGROUND

Wound dressings are used to facilitate healing and prevent infection as wound care products. Most the dressing on the market focus on controlling moisture levels or preventing infection though physical barriers or additives. Most additives to woven and non-woven dressings are antimicrobials, often dispersed in an ointment or as a nanoparticle.

Bioglass has been investigated and various formulations patented since the late 1960s when it was first introduced by Hench. The initial work has focused largely on bioglass, or bioactive glass, for bone tissue engineering or treatment of bone defects. Bioglass has been shown to form hydroxyapatite in the presence of simulated body fluid, and in bone tissue applications. The ionic conversion of the glass at the interface is thought to assist in the hydroxyapatite formation characteristic of bioactive glasses. There is a need to provide better dressings in a form useful for treating wounds.

### SUMMARY

Thus, in one aspect, the present disclosure provides an article, comprising: a nonwoven matrix; and bioactive glass particles enmeshed in the nonwoven matrix; wherein the article is a conformable wound dressing.

In another aspect, the present disclosure provides a method of making the article of present disclosure, comprising: flowing molten polymer through a plurality of orifices to form filaments; attenuating the filaments into fibers; directing a stream of bioactive glass particles amidst the filaments or fibers; and collecting a nonwoven matrix and bioactive glass particles enmeshed in the nonwoven matrix.

Various aspects and advantages of exemplary embodiments of the present disclosure have been summarized. The above Summary is not intended to describe each illustrated embodiment or every implementation of the present disclosure. Further features and advantages are disclosed in the embodiments that follow. The Detailed Description that follows more particularly exemplify certain embodiments using the principles disclosed herein.

### DETAILED DESCRIPTION

Before any embodiments of the present disclosure are explained in detail, it is understood that the invention is not limited in its application to the details of use, construction, and the arrangement of components set forth in the following description. The invention is capable of other embodiments and of being practiced or of being carried out in various ways that will become apparent to a person of ordinary skill in the art upon reading the present disclosure. Also, it is understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is understood that other embodiments may be utilized, and structural or logical changes may be made without departing from the scope of the present disclosure.

An article suitable for use as a wound dressing is described. The article includes a nonwoven matrix and bioactive glass particles enmeshed in the nonwoven matrix. In some embodiments, the article is a conformable wound dressing. A conformable wound dressing means that the dressing can be shaped to the contours of a wound bed, enabling interaction of the dressing with a non-uniform surface found in different kinds of wounds.

The nonwoven can be a nonwoven web which may include nonwoven webs manufactured by any of the commonly known processes for producing nonwoven webs. In some embodiments, the nonwoven matrix can be a nonwoven fibrous web of polymeric fibers. As used herein, the term “nonwoven fibrous web” refers to a fabric that has a structure of individual fibers or filaments which are randomly and/or unidirectionally interlaid in a mat-like fashion. For example, the fibrous nonwoven web can be made by carded, air laid, wet laid, spunlaced, spunbonding, electrospinning or melt-blowing techniques, such as melt-spun or melt-blown, or combinations thereof. Spunbonded fibers are typically small diameter fibers that are formed by extruding molten thermoplastic polymer as filaments from a plurality of fine, usually circular capillaries of a spinneret with the diameter of the extruded fibers being rapidly reduced. Melt-blown fibers are typically formed by extruding the molten thermoplastic material through a plurality of fine, usually circular, die capillaries as molten threads or filaments into high velocity, usually heated gas (e.q. air) stream which attenuates the filaments of molten thermoplastic material to reduce their diameter. Thereafter, the meltblown fibers are carried by the high velocity gas stream and are deposited on a collecting surface to form a web of randomly distributed meltblown fibers. Any of the non-woven webs may be made from a single type of fiber or two or more fibers that differ in the type of thermoplastic polymer and/or thickness. In some embodiments, a plurality of randomly oriented discrete fibers can be entangled to form the nonwoven fibrous web. In some embodiments, bioactive glass particles are not substantially bonded to the fibers. In some embodiments, less than 50%, 40%, 30%, 20%, 15%, 10%, 9%, 8%, 7%, 6%, 5%, 4%, 3%, 2% or 1% bioactive glass particles are bonded to the fibers.

Thermoplastic material can include polyurethane elastomer, polybutylene elastomer, polyester elastomer, a hydrogenated styrene isoprene/butadiene styrene block copolymer(s). Suitable polyolefins for making the nonwoven web include, but are not limited to, polyethylene, polypropylene, poly(1-butene), copolymers of ethylene and propylene, alpha olefin copolymers (such as copolymers of ethylene or propylene with 1-butene, 1-hexene, 1-octene, and 1-decene), poly(ethylene-co-1-butene), poly(1-methylpentene) and poly(ethylene-co-1-butene-co-1-hexene).

Further details on the manufacturing method of nonwoven webs of this invention may be found in Wente, Superfine Thermoplastic Fibers, 48 INDUS. ENG. CHEM. 1342(1956), or in Wente et al. Manufacture of Superfine Organic Fibers, (Naval Research Laboratories Reort No. 4364, 1954). Useful methods of preparing the nonwoven substrates are described in U.S. RE39,399 (Allen), U.S. Pat. No. 3,849,241 (Butin et al.), U.S. Pat. No. 7,374,416 (Cook et al.), U.S. Pat. No. 4,936,934 (Buehning), and U.S. Pat. No. 6,230,776 (Choi).

Bioactive glass used in the invention may be melt-derived or sol-gel derived. A bioactive glass material suitable for the present articles and methods may have silica, sodium, calcium, phosphorous, and boron present, as well as combinations thereof. In some embodiments, sodium, boron, phosphorous and calcium may each be present in the compositions in an amount of about 1% to about 99%, based on the weight of the bioactive glass. In further embodiments, sodium, boron, phosphorous and calcium may each be present in the composition in about 1%, about 2%, about 3%, about 4%, about 5%, about 6%, about 7%, about 8%, about 9%, or about 10%. In certain embodiments, silica, sodium, boron, and calcium may each be present in the composition in about 5 to about 10%, about 10 to about 15%, about 15 to about 20%, about 20 to about 25%, about 25 to about 30%, about 30 to about 35%, about 35 to about 40%, about 40 to about 45%, about 45 to about 50%, about 50 to about 55%, about 55 to about 60%, about 60 to about 65%, about 65 to about 70%, about 70 to about 75%, about 75 to about 80%, about 80 to about 85%, about 85 to about 90%, about 90 to about 95%, or about 95 to about 99%. Some embodiments may contain substantially one or two of sodium, calcium, phosphorous, and boron with only traces of the other(s). The term “about” as it relates to the amount of calcium phosphate present in the composition means+/−0.5%. Thus, about 5% means 5+/−0.5%. Divalent cations or ions that may be present in any of the bioactive glasses of this and other aspects of the invention include one or more of iron-II, iron-III, alumina, cobalt, copper, magnesium, and zinc. Strontium, Potassium, Fluorine. Silver, barium, titanium

The bioactive glass materials may further comprise one or more of a silicate, borosilicate, borate, or calcium, including CaO, P2O₅, SiO₂, and B₂O₃. An exemplary bioactive glass is 45S5, which includes 46.1 mol % SiO₂, 26.9 mol % CaO, 24.4 mol % Na2O and 2.5 mol % P2O₅. An exemplary borate bioactive glass is 45S5B1, in which the SiO2 of 45S5 bioactive glass is replaced by B2O3. Other exemplary bioactive glasses include 58S, which includes 60 mol % SiO2, 36 mol % CaO and 4 mol % P₂O₅, and S70C30, which includes 70 mol % SiO₂ and 30 mol % CaO. Other exemplary bioactive glasses include PSr40 which is 50% P₂O₅, 40% SrO, 10% Na₂O Mol %.

The bioactive glass may comprise one or more of SiO₂, CaO, Na₂O, P₂O₅, K₂O, MgO, and B₂O₃. The bioactive glass may comprise CaO, Na₂O, and P₂O₅.

The following composition, having a weight % of each element in oxide form in the range indicated, will provide one of several bioactive glass compositions that may be used to form a bioactive glass:

| | |
|---|---|
| SiO₂ | 0-86 |
| CaO | 4-35 |
| Na₂O | 0-35 |
| P₂O₅ | 2-15 |
| CaF₂ | 0-25 |
| B₂O₃ | 0-75 |
| K₂O | 0-8 |
| MgO | 0-5 |
| CaF | 0-35 |

In some embodiments, the article is capable of releasing ion, for example, calcium ion from the bioactive glass particles. Calcium ion can play a role in enzyme and protein activity levels. Ca ion levels are particularly critical to Epithelial Cadherin (e-cadherin), a poly peptide which mediates cell to cell adhesion and recognition, allowing cell migration into the wound bed.

In some embodiments, the weight % of bioactive glass in the conformable matrix is greater than 3 weight %, greater than 5 weight %, greater than 10 weight %, greater than 15 weight %, greater than 20 weight %, greater than 30 weight %, greater than 40 weight %, greater than 50 weight %, greater than 60 weight %, greater than 70 weight %, greater than 80 weight %, or greater than 90 weight %.

In some embodiments, the weight % of bioactive glass in the conformable matrix is less than 95 weight %, less than 90 weight %, less than 80 weight %, less than 70 weight %, less than 60 weight %, less than 50 weight %, less than 45 weight %, less than 40 weight %, less than 35 weight %, less than 30 weight %, less than 25 weight %, less than 20 weight %, less than 15 weight %, or less than 10 weight %.

In some embodiments, the weight % of bioactive glass in the conformable matrix is about 10-95 weight %, about 10-80 weight %, about 50-70 weight %, about 70-95 weight %. about 60-80 weight %, about 3-50 weight %, about 3-35 weight %, about 3-25 weight %, about 5-50 weight %, about 5-35 weight %, about 5-25 weight %, about 10-50 weight %, about 10-35 weight %, about 10-25 weight %, about 25-50 weight %, about 35-50 weight %, about 3-10 weight %, about 10-20 weight %, about 20-30 weight %, about 30-40 weight %, about 40-50 weight %, about 5-35 weight %, about 5-30 weight %, about 5-15 weight %, about 15-25 weight %, about 25-35 weight %, or about 35-45 weight %.

In some embodiments, the article can include a substrate. The substrate can be selected from foam, mesh, netting, woven, nonwoven, cotton, cellulose fabrics, perforated film, hydrocolloid, hydrogel, polymers with inherent porosity, pressure sensitive adhesive and combination of thereof. In some embodiments, the substrate can be an absorbent substrate selected from foam, mesh, netting, woven, nonwoven, cotton, cellulose fabrics, perforated film, hydrocolloid, hydrogel, polymers with inherent porosity, pressure sensitive adhesive and combination of thereof. Exemplary absorbent substrate can include film, fabrics or porous article made from viscose, rayon, alginate, gauze, biopolymers, polyurethane, biodegradable polymers or the polymers described in U.S. Pat. No. 7,745,509, the disclosures of which is hereby incorporated by reference.

The absorbent materials used in the absorbent substrate can be manufactured of any suitable materials including, but not limited to, woven or nonwoven cotton or rayon or netting and perforated film made from nylon, polyester or polyolefins. Absorbent pad can be used as the absorbent layer and can be useful for containing a number of substances, optionally including drugs for transdermal drug delivery, chemical indicators to monitor hormones or other substances in a patient, etc.

The absorbent layer may include a hydrocolloid composition, including the hydrocolloid compositions described in U.S. Pat. Nos. 5,622,711 and 5,633,010, the disclosures of which are hereby incorporated by reference. The hydrocolloid absorbent may comprise, for example, a natural hydrocolloid, such as pectin, gelatin, or carboxymethylcellulose (CMC) (Aqualon Corp., Wilmington, Del.), a semi-synthetic hydrocolloid, such as cross-linked carboxymethylcellulose (X4ink CMC) (e.g. Ac-Di-Sol; FMC Corp., Philadelphia, Pa.), a synthetic hydrocolloid, such as cross-linked polyacrylic acid (PAA) (e.g., CARBOPOL™ No. 974P; B.F. Goodrich, Brecksville, Ohio), or a combination thereof. Absorbent layer can be manufactured of other synthetic and natural hydrophilic materials including polymer gels and foams. In one embodiment the substrate is a hydrocolloid polymer.

The article can be in any suitable physical form, such as a sheet (i.e. film), foam sheet, or nonwoven matrix disposed on or within a carrier layer. For example, the nonwoven matrix can be disposed on or within a carrier. In some embodiments, the carrier can be a carrier layer disposed on a major surface of the article. A carrier layer is typically disposed on the opposing major surface as the wound-facing surface.

In some embodiments, carrier layer is a release liner. The release liner carrier may be disposed on the opposing major surface of both major surfaces (not shown) such that the nonwoven matrix is between the release liner layers.

Various release liners are known such as those made of (e.g. kraft) papers, polyolefin films such as polyethylene and polypropylene, or polyester. The films are preferably coated with release agents such as fluorochemicals or silicones. For example, U.S. Pat. No. 4,472,480 describes low surface energy perfluorochemical liners. Examples of commercially available silicone coated release papers are POLYSLIK™, silicone release papers available from Rexam Release (Bedford Park, Ill.) and silicone release papers supplied by LOPAREX (Willowbrook, Ill.). Other non-limiting examples of such release liners commercially available include siliconized polyethylene terephthalate films commercially available from H. P. Smith Co. and fluoropolymer coated polyester films commercially available from 3M under the brand “ScotchPak™” release liners.

In other embodiments, the carrier layer may comprise a variety of other (e.g. flexible and/or conformable) carrier materials such as polymeric films and foams as well as various nonwoven and woven fibrous materials, such as gauze. In some embodiments, the carrier layer is absorbent, such as an absorbent foam. In other embodiments, the carrier layer is non-absorbent, such as a polymeric film.

In some embodiments, the article has a pH value of more than 5, 6, 7, 8, 9, or 10 as determined by Method A of the current application described herein. In some embodiments, the article has a pH value of about 5 to about 11.5, about 5 to about 11, about 6 to about 11.5, about 7.5 to about 11.5, about 8 to about 11.5, or about 6 to about 6.8 as determined by Method A.

Method A is described as follows: The nonwoven web (10 mg/mL) is immersed in distilled water. The water is maintained at 25° C. with the nonwoven web completely immersed in the water. The pH of the distilled water before the addition of the nonwoven web is 6.8-7.2. Following immersion for 24 hours, the pH value of the water is measured using a calibrated pH meter (Accumet AE150 pH Benchtop Meter, obtained from Thermo Fisher Scientific, Waltham, MA).

In some embodiments, the article has a pH value of less than 12, 11.5, 11, 10, 9, 8, or 7 determined by Method A. Not to be bound by theory, altering pH may help facilitate the recovery of wound tissue by reducing enzymatic activity

In some embodiments, a method of forming an article of the current application is described. The method can include flowing molten polymer through a plurality of orifices to form filaments, attenuating the filaments into fibers, directing a stream of bioactive glass particles amidst the filaments or fibers; and collecting a nonwoven matrix and bioactive glass particles enmeshed in the nonwoven matrix. In some embodiments, the filaments can be melt-blown to form the nonwoven matrix. The polymer to form the nonwoven matrix can include any polymer described above, for example, a polyurethane elastomer, a polybutylene elastomer, a polyester elastomer, polyolefin, for example, polypropylene/polyethylene or a hydrogenated styrene isoprene/butadiene styrene block copolymer(s). In some embodiments, the nonwoven matrix comprises water soluble fibers, absorbent fibers or elastic fibers. In some embodiments, the nonwoven matrix comprises polyolefin fibers.

In some embodiments, the method may further comprise adding the matrix on a substrate. In some embodiments, the method may further comprise disposing the conformable matrix on or within a carrier.

In some embodiments, a method of treating a wound with an article of the current application is described. The method of treatment involves covering at least a portion of the wound with the article. In some embodiments, the method of treatment increases the pH of the wound environment. In some embodiments, the method of treatment decreases the pH of the wound environment. The wound to be treated by the method can be an open wound of the skin that exposes underlying body tissue. Open wounds that can be treated by the method include acute wounds and chronic wounds. Open wounds that can be treated by the method include wounds to the skin from trauma (for example avulsions, incisions, and lacerations); wounds to the skin from pressure (for example pressure ulcers); and wounds to the skin from disease (for example venous ulcers, diabetic foot ulcers, and diabetic leg ulcers).

The following working examples are intended to be illustrative of the present disclosure and not limiting.

### EXAMPLES

Objects and advantages of this invention are further illustrated by the following examples, but the particular materials and amounts thereof recited in these examples, as well as other conditions and details, should not be construed to unduly limit this invention.

Bioactive Glass (BG) was obtained from the 3M Corporation (St. Paul, MN) as a powder with the following composition: SiO₂(45 weight %), Na₂O (24.5 weight %), CaO (24.5 weight %), P₂O₅(6 weight %). The reported glass transition temperature (Tg) was 528.6° C.

The weight percent (wt. %) of bioactive glass (BG) enmeshed in the nonwoven web was determined by comparing the basis weight (BW) of the nonwoven web containing enmeshed bioactive glass to the basis weight of web prepared under the same conditions in which no bioactive glass particles were fed into the apparatus (Equation 1).

$$wt . \% of B G = [ 1 - [ \frac{B W of Nonwoven web with No added B G}{B W of Nonwoven web with Enmeshed B G} ] ] \times 100 \qquad Equation 1$$

### Example 1

An apparatus as described in FIG. 6 of US Patent Application 20060096911 (Brey) was used to prepare the meltblown nonwoven webs containing enmeshed bioactive glass particles. The polymer used was KRATON MD1648 polymer (a styrene-ethylene-butylene-styrene (SEBS) polymer obtained from the KRATON Corporation, Houston, TX). The polymer melt temperature was set at 225° C. and the heated air stream used to attenuate filaments into fibers was set at 355° C. The die to collector distance was about 55.9 cm. Bioactive glass powder was added at a variable feed rate to prepare nonwoven webs with either a 20 wt. %, 49 wt. %, or 74 wt. % loading of bioactive glass in the web. The speed of the collector was adjusted so that the finished nonwoven web with enmeshed bioactive glass had a basis weight of about 500 gsm (grams per square meter). The average fiber diameter was about 19 micrometers.

### Example 2

The same procedure as described in Example 1 was followed with the exception that the polymer used was IGORAN PS 440-200 polymer (a polyester-based thermoplastic polyurethane (TPU) polymer obtained from the Huntsman Corporation, The Woodlands, TX). The polymer melt temperature was set at 225° C. and the heated air stream used to attenuate filaments into fibers was set at 355° C. The die to collector distance was about 55.9 cm. Bioactive glass powder was added at a variable feed rate to prepare nonwoven webs with either a 34 wt. %, 40 wt. %, 45 wt. %, or 74 wt. % loading of bioactive glass in the web. The speed of the collector was adjusted so that the finished nonwoven web with enmeshed bioactive glass had a basis weight of about 480 gsm. The average fiber diameter was about 17 micrometers. For the web with a bioactive glass loading of 34 wt. %, the die to collector distance was about 38.1 cm.

### Example 3

The same procedure as described in Example 1 was followed with the exception that the polymer used was METOCENE MF650Y polymer (a crystalline polypropylene polymer obtained from LyondellBasell, Houston, TX). The polymer melt temperature was set at 250° C. and the heated air stream used to attenuate filaments into fibers was set at 380° C. The die to collector distance was about 25.4 cm. Bioactive glass powder was added at a variable feed rate to prepare nonwoven webs with either a 10 wt. %, 20 wt. %, 31 wt. %, 41 wt. %, or 55 wt. % loading of bioactive glass in the web. The speed of the collector was adjusted so that the finished nonwoven web with enmeshed bioactive glass had a basis weight of about 200 gsm. The average fiber diameter was about 3 micrometers. As a control, a nonwoven web was prepared in which no bioactive glass was added. The finished articles were tested for pH according to Method A (described above) and the results are reported in Table 1.

TABLE 1

| Bioactive Glass Enmeshed in the | |
|---|---|
| Nonwoven Article (wt. %) | pH at 24 Hours |
| 0 (control) | 7.4 |
| 10 | 9.9 |
| 20 | 10.3 |
| 31 | 10.5 |
| 41 | 10.5 |
| 55 | 10.7 |

All references and publications cited herein are expressly incorporated herein by reference in their entirety into this disclosure. Illustrative embodiments of this invention are discussed and reference has been made to possible variations within the scope of this invention. For example, features depicted in connection with one illustrative embodiment may be used in connection with other embodiments of the invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. An article, comprising:
a nonwoven matrix; and
bioactive glass particles enmeshed in the nonwoven matrix;
wherein the article is a conformable wound dressing.

2. The article of claim 1, wherein the article has a pH value from 5 to 11.

3. The article of claim 1, wherein the article comprises 10-80 wt % bioactive glass particles.

4. The article of claim 1, wherein the nonwoven matrix is a nonwoven fibrous web of polymeric fibers.

5. The article of claim 4, wherein a plurality of randomly oriented discrete fibers is entangled to form the nonwoven fibrous web.

6. The article of claim 5, wherein bioactive glass particles are not substantially bonded to the fibers.

7. The article of claim 1, wherein the article is capable of releasing ion, including calcium ion.

8. The article of claim 1, wherein the article comprises a substrate.

9. The article of claim 1, further comprising a carrier wherein the conformable matrix is disposed on or within the carrier.

10. A method of making the article of claim 1, comprising:
flowing molten polymer through a plurality of orifices to form filaments;
attenuating the filaments into fibers;
directing a stream of bioactive glass particles amidst the filaments or fibers; and
collecting a nonwoven matrix and bioactive glass particles enmeshed in the nonwoven matrix.

11. The method of claim 10, further comprising meltblowing the filaments.

12. The method of claim 1, wherein the nonwoven matrix comprises water soluble fibers, absorbent fibers or elastic fibers.

13. The method of claim 1, wherein the nonwoven matrix comprises polyolefin fibers.

14. The method of claim 1, wherein the molten polymer comprises a polyurethane elastomer, a polybutylene elastomer, a polyester elastomer, a polypropylene/polyethylene, or a hydrogenated styrene isoprene/butadiene styrene block copolymer(s).

15. The method of claim 1, wherein 10-80 wt % bioactive glass particles are enmeshed in the nonwoven matrix.
